(19) United States
(12) Patent Application Publication
(10) Pub. No.: US 2024/0287464 A1
(43) Pub. Date: Aug. 29, 2024

(54) A MEDIUM FOR STORING ISOLATED PANCREATIC ISLETS AND A METHOD FOR STORING ISOLATED PANCREATIC ISLETS

(71) Applicant: POLBIONICA SP. Z O.O., Warszawa (PL)

(72) Inventors: Michal WSZOLA, Warszawa (PL); Marta KLAK, Pila (PL); Andrzej BERMAN, Warszawa (PL); Tomasz BRYNIARSKI, Nowy Targ (PL); Katarzyna KOSOWSKA, Jastrzab (PL)

(73) Assignee: POLBIONICA SP. Z O.O., Warszawa (PL)

(21) Appl. No.: 18/573,713

(22) PCT Filed: Jun. 30, 2021
(86) PCT No.: PCT/PL2021/050051

§ 371 Date: Dec. 22, 2023

(51) Int. Cl. C12N 5/071; A01N 1/02

(52) U.S. Cl. CPC C12N 5/0676; A01N 1/0231; C12N 2533/52; C12N 2533/54; C12N 2533/80; C12N 2533/90

(57) ABSTRACT

The invention relates to a medium for storing isolated pancreatic islets and a method for storing isolated pancreatic islets. Such a medium is suitable for storing pancreatic islets both at physiological temperature and refrigerated.

### FIELD OF INVENTION

The invention relates to a supplemented medium for storing isolated pancreatic islets and to a method for storing isolated pancreatic islets. Such a medium is suitable for storing pancreatic islets both at physiological temperature and refrigerated.

### PRIOR ART

Numerous media are known in the art for storing isolated cells, tissues and organs. Such media contain various components such as extracellular matrix (ECM), protein additives and the like.

Document KR1020170011676A discloses a method for differentiating stem cells into hepatocytes using a concentrate derived from decellularized biological tissue (ECM), wherein the ECM may be derived from mammalian liver, heart, kidney, stomach, small intestine, colon, spleen, bladder, lung or skin. Authors note that the ECM mimics cell-specific microenvironment in vivo, and that obtaining and using natural extracellular matrix from specific tissues can provide an optimal growth environment. They also note that using a combination of single proteins of collagen, fibronectin, vitronectin, laminin or matrigel will not achieve such a result. In the embodiments, liver-derived ECM (LECM) were added to the medium at 2, 5, 10 and 20%.

Document WO2019185017A relates to a medium for culturing liver cells and organoids based on a base medium supplemented with L-glutamine and a pH regulator. In order to improve their survival, hepatocytes are contacted with ECM, wherein the ECM may be of natural or commercial origin (e.g. Matrixgel TM, BD Biosciences).

Document BR112013002249A2, in turn, relates to culturing stem cells derived from a liver fragment in contact with ECM (Matrigel) in a base medium supplemented with epidermal growth factor (EGF), Wnt agonist, fibroblast growth factor (FGF) and nicotinamide.

Another document, US20080241919A1, describes a system for culturing embryonic stem cells comprising a vessel for culturing cells that comprises a medium containing ECM, wherein the ECM may contain laminin, fibronectin, collagen and gelatin. ECM components may also be present in the solution. The medium also contains bFGF, insulin and ascorbic acid.

Document U.S. 20030026788A1 describes the use of pieces of extracellular matrix (ECM), including an ECM harvested from tissue donors to preserve the unique characteristics of cultured cells.

Document AU2002314621A1 relates to a method for culturing clusters of cells (islets of Langerhans) isolated from an organ with vascular tissue in a medium containing an ECM component, which may be laminin and/or type IV collagen.

Document U.S. Pat. No. 5,985,539A relates to a method for reconstructing an animal organ comprising perfusing the organ through a vascular system with a cell culture medium solution comprising an ECM component (collagen, fibronectin, vitronectin, laminin, proteoglycan or glycosaminoglycan). Examples include use of 0.25% type I collagen.

Document EP0703978A1 describes a serum-free or serum-depleted medium for short-and long-term proliferation and growth of cells, in particular haematopoietic and bone marrow stromal cells. The medium is based on a standard culture medium such as Iscove's modified Dulbecco medium. Components of an extracellular matrix such as collagen (preferably types I and IV), fibronectin and laminin are used for cell adhesion on a surface.

Document U.S. 20080248570A1 discloses a method for proliferating liver cells, including liver precursors ex vivo on or in hyaluronates with or without other extracellular matrix components (such as collagens I, III and IV, basic adhesion molecules, proteoglycans or glycosaminoglycans thereof) and with or without hormones and/or growth factors. Compositions containing ECM have also been disclosed. Examples disclose the use of 1% hyaluronic acid gel.

Document U.S. 20110150823A1 discloses a gel containing collagen and hyaluronate in a weight ratio of 0.01-100:1 and its use for culturing stem cells. In the embodiments, cells were resuspended in low glucose Dulbecco's Modified Eagle's Medium (Gibco BRL, Life Technologies) supplemented with 20% fetal bovine serum and 200 U/mL gentamycin, at a concentration of 10⁵-10⁷ cells/mL, and mixed with various amounts of hyaluronate to form a suspension. Collagen was obtained from pig dermis and dissolved in 0.01 N HCl to produce a collagen solution, pH 4-7. The collagen solution was mixed with a suspension containing stem cells in an equal volume to form a mixture containing 6 mg/mL collagen and 0.05 mg/mL, 0.2 mg/mL or 0.5 mg/mL hyaluronan or 9 mg/mL collagen and 0.2 mg/mL hyaluronan.

Document U.S. 20070155009A1 describes a method for proliferating hepatic progenitor cells in vitro comprising: (a) providing isolated hepatic progenitor cells and (b) culturing isolated liver-derived progenitor cells on a layer of extracellular matrix components from a liver. The extracellular matrix component may be selected from a group consisting of type III collagen, type IV collagen, laminin, hyaluronan or a combination thereof.

Document WO2013116446A1 relates to smooth muscle cultures in collagen or collagen-laminin suspensions.

Document CN1816279B relates to a medium for preserving organs, biological tissues or living cells, containing a liquid nutrient base, characterised in that it contains high molecular weight hyaluronic acid and sodium chloride and does not contain an ingredient of animal origin. The medium contains 80-4000 mg/l, preferably 100-200 mg/l, preferably 100-160 mg/l of high molecular weight hyaluronic acid. The medium may be based on CMRL1066 medium.

Document WO2007009981A1 describes a tissue culture medium based on DMEM/F12 medium supplemented with e.g. hyaluronic acid at 50 mg/L.

Document CN112608899A relates to a use of serum medium in spheroidal tumour tissue culture. A collagen gel based on F-12 medium with 1.3 mg/L collagen was also used for the culture.

Document JP1993184356A discloses a medium and a method for culturing invertebrate nervous tissue. A target medium is composed of a collagen gel containing (A) L-15 medium, (B) inactivated serum, (C) glucose, and (D) an antibiotic substance. Preferably, amount of component B is 10-20% by weight relative to the medium, and amount of component C preferably is 10-20 mg/mL. The collagen content of the medium is preferably 0.1-0.3% by weight.

Document U.S. Pat. No. 7,550,294B2 discloses a method for culturing cells, tissues and organs in vivo in a medium based on CMRL1066, while document JP1988146785A describes a culture of animal cells in a medium (e.g. CMRL1066) supplemented with L-carnitine.

### OBJECT OF THE INVENTION

The object of the invention is to develop a medium to allow for storing isolated pancreatic islets, both at physiological temperature and refrigerated, with no loss of their secretory properties. This was achieved by adding extracellular matrix (ECM) or isolated proteins thereof in appropriate proportions to a commercial medium.

The medium for storing isolated pancreatic islets according to the invention is based on a commercial medium CMRL 1066 or RPMI and comprises an additive of extracellular matrix (ECM) proteins.

Preferably, the base is the commercial medium CMRL 1066.

Preferably, the additive of ECM proteins comprises the following proteins in appropriate amounts by weight per 1L of medium:
laminin in an amount of 67 to 100 mg/L of medium, preferably 84.3 mg/L of medium
hyaluronic acid in an amount of 5 to 8 mg/L of medium, preferably 6.7 mg/L of medium
collagen I in an amount of 32 to 48 mg/L of medium, preferably 40.7 mg/L of medium
collagen IV in an amount of 100 to 145 mg/L of medium, preferably 122.3 mg/L of medium

Preferably, the additive of ECM proteins comprises the following proteins in appropriate % by volume relative to the volume of the medium:
laminin 6.74-10.12%, preferably 8.43% by volume of the medium,
hyaluronic acid 0.10-0.16%, preferably 0.13% by volume of the medium,
collagen I 3.26-4.88%, preferably 4.07% by volume of the medium; and
collagen IV 9.78-14.67%, preferably 12.23% by volume of the medium,
   wherein laminin at a starting concentration of 1 mg/mL, hyaluronic acid at a concentration of 5 mg/mL, collagen I at a concentration of 1 mg/mL, and collagen IV at a concentration of 1 mg/mL are added to the medium

Preferably, the hyaluronic acid may be a high or low molecular weight hyaluronic acid.

Preferably, a detergent-free dECM solution, preferably of animal origin, is used as the additive of ECM proteins.

Preferably, the medium comprises 1% to 2.5% by volume, preferably 1%, of the additive of ECM proteins.

Preferably, the medium further comprises a cryoprotectant and/or an antibiotic and/or a fungicide and/or glucose and/or a serum and/or an amino acid.

Preferably, the medium comprises L-glutamine, preferably in an amount of 5 mL for each 500 mL of base medium, 100× Penicillin-streptomycin, preferably in an amount of 5 mL for each 500 mL of base medium, amphotericin B, preferably in an amount of 5 mL for each 500 mL of base medium, glucose, preferably in an amount equivalent to 10 mL of 280 mM glucose solution for each 500 mL of base medium, and FBS, preferably in an amount of 50 mL for each 500 mL of base medium.

A method for storing isolated pancreatic islets according to the invention is characterised in that isolated pancreatic islets are stored in the medium according to the invention, at a temperature between 4 and 37° C., preferably at 4° C.

Preferably, before the islets are placed in the medium, the islets are treated or they remain untreated

Preferably, the additive of ECM proteins in the form of a dECM solution, constituting an ingredient of the medium, is obtained by a method according to patent application EP19218191.

### BRIEF DESCRIPTION OF FIGURES

The object of the invention in the embodiment is shown in the drawing, where:

FIGS. 1-3 are graphs showing functionality of untreated and treated pancreatic islets after 24 h of islet incubation at a temperature of 4° C. and 37° C., wherein medium contained CMRL1066 medium enriched with 1 and 2.5% detergent-free ECM, and control samples were incubated in CMRL1066 medium;

FIG. 4-6 are graphs showing functionality of untreated and treated pancreatic islets after 48 h of islet incubation at a temperature of 4° C. and 37° C., wherein medium contained CMRL1066 medium enriched with 1 and 2.5% detergent-free ECM, and control samples were incubated in CMRL1066 medium;

FIG. 7 is a graph showing functionality of untreated and treated pancreatic islets after 72 hours of islet incubation at a temperature of 4° C. and 37° C., wherein medium contained CMRL1066 medium enriched with 1 and 2.5% detergent-free ECM, and control samples were incubated in CMRL1066 medium;

FIG. 8 is a graph showing functionality of untreated pancreatic islets after 96 hours of islet incubation at a temperature of 4° C. and 37° C., wherein medium contained CMRL1066 medium enriched with 1 and 2.5% detergent-free ECM;

FIG. 9 is a graph showing functionality of untreated pancreatic islets after 120 hours of islet incubation at a temperature of 4° C., wherein medium contained CMRL 1066 medium enriched with 1 and 2.5% detergent-free ECM-B2-ECM 1%-untreated, 4° C., E1-untreated control, 4° C., E2-ECM 2.5% untreated, 4° C.

FIG. 10 is a graph showing functionality of untreated pancreatic islets after 72 hours of islet incubation at a temperature of 4° C., wherein medium contained CMRL 1066 medium enriched with the commercial additives laminin, hyaluronic acid, collagen I and IV;

In the figures, lower axis represents time points every 15 minutes: 1—low glucose control point, 2, 3—measurement after 15 at 30 min in low glucose, 4—high glucose control measurement, 5-8—measurement at 15, 30, 45 and 60 min in high glucose, 9—low glucose control point, 10-13—measurement at 15, 30, 45 and 60 min in low glucose.

The medium according to the present invention is to be used to store isolated pancreatic islets. Said medium is suitable for storing pancreatic islets both under physiological temperature conditions, i.e. at 37° C., and in a refrigerated state-at a standard refrigeration storage temperature, i.e. at 4° C.

According to literature data presented by Llacua et al. dddCollagen type VI interaction improves human islet survival in immunoisolating microcapsules for treatment of diabetes. ISLETS (2018), vol. 10, no. 2, 60-68) when isolating pancreatic islets, ECM particles that surround the islets and connect endocrine cells to each other are damaged as a result of adding collagenases. This adversely impacts both laminins and collagens types I, III, IV, V and VI which are components of the pancreatic islets, and ultimately damages the entire ECM structure. Supplementation of media with externally supplied ECM or ECM components enhances survival and allows pancreatic islets to retain their function. Cells can grow because the conditions provided by such a medium, including biochemical composition, fibrous structure and viscoelastic properties, are closer to those in target organ.

For the purposes of the present invention, extracellular matrix (ECM) proteins defines ECM, preferably of animal origin (e.g. derived from pig, cow, sheep, etc.), preferably decellularized/cell-free (dECM) ECM, preferably obtained using a method described in European patent application EP19218191, or a mixture of commercially available ECM proteins, i.e. laminin, hyaluronic acid, collagen I and collagen IV in appropriate proportions.

In a preferred embodiment, respective protein components of the ECM in the medium are as follows:
laminin 8.43% by volume of the medium,
hyaluronic acid 0.13% by volume of the medium,
collagen I 4.07% by volume of the medium; and
collagen IV 12.23% by volume of the medium,
   wherein laminin at a starting concentration of 1 mg/mL, hyaluronic acid at a concentration of 5 mg/mL, collagen I at a concentration of 1 mg/mL, and collagen IV at a concentration of 1 mg/mL are added to the medium.

The hyaluronic acid used in the mixture according to the present invention may be either a high-or a low molecular weight acid.

The medium may also contain other optional ingredients such as cryoprotectants, antibiotics, fungicides, glucose, serum (FBS), amino acids added at concentrations usually used in the art, e.g. the medium may contain FBS, D-glucose, a mixture of antibiotics (penicillin, streptomycin, amphotericin B, cephalosporin) and L-glutamine.

The medium according to the invention is obtained by mixing the ingredients as above in appropriate proportions. Mixing can be performed manually with a dipstick or automatically with any suitable stirrer, such as a magnetic stirrer. Preferably, regardless of selected method, the mixing is performed under sterile conditions.

According to the invention, the word “approximately” used above and below is to be understood as a deviation of +/−5% from the indicated value, reflecting inaccuracies which may occur in a course of preparation process of the composition according to the invention, e.g. in the course of measuring the ingredients.

### EMBODIMENTS

In order to implement the following embodiments, a number of procedures were performed in order to obtain the test materials. Since these procedures are common to all embodiments, they will be described before describing the examples themselves.

### 1) Harvesting the Material

Pancreases were harvested from breeding pigs. First step of pancreas harvesting was to cut pancreatic duct near duodenal ostium and to retrogradely inject 20 mL of ice-cold preservation solution UW into it. After the lavage, the pancreas was separated from surrounding tissues using scissors and placed in a sterile bag with 500 mL of ice-cold UW preservation solution and then transferred to an isolation laboratory in a container with crushed ice. Total mean warm ischaemia time was 10 min.

### 2) Isolation of Pancreatic Islets

Isolation of pancreatic islets was started directly after the organ was delivered to the isolation laboratory. In a first step, the pancreas was subjected to a three-step decontamination process. In order to decontaminate the organ, the pancreas was immersed in an ice-cold solution of 5% betadine in 1× PBS for 5 min. The pancreas was then washed twice with cold antibiotic solution (Penicillin/Streptomycin—10,000 U/10,000 U in 1×PBS). In a next step, the organ was mechanically cleaned by removing remaining blood vessels, lymph nodes as well as fatty tissue. The cleaning process was carried out under hypothermic conditions. Then, after weighing the organ, it was injected via a catheter inserted proximally into pancreatic duct with an ice-cold solution of collagenase NB8 (2.73 mg collagenase/g pancreas) and neutral protease (100 DMC-U) (150 mL Ringer's fluid with pH 7.2 to 7.4, 18 mM HEPES, 7 mM CaCl₂ and 0.05% NaHCO₃). The fluid was administered through Wirsung tube at a rate of approximately 5 mL/min. The pancreas was weighed, mechanically fragmented and placed in a Ricordi chamber. The system was then filled with one litre of Priming Solution (1.25% nicotinamide, 0.5% glucose, 0.025% NaHCO₃, heated to 37° C., 14 mM HEPES, 5 mM sodium pyruvate and 44U/44U Penicillin/Streptomycin in Ringer's fluid with pH 7.2 to 7.4). Pancreatic digestion was mechanically assisted by shaking the Ricordi chamber with beads placed therein. Progress of the digestion was monitored by taking samples of working fluid from the system every 30-60 s, staining them with 0.1% dithizone and quantifying the islets under a microscope. When a large quantity of islets appeared in the field of view, digestion was quenched (arbitrary assessment; mean digestion time was 19 min. and 40 s.—there were no statistical differences between groups) by adding approximately 200 mL of FBS to the system and placing the cooler in ice. Priming Solution was then replaced with 5 l Dilution Solution (1.2% nicotinamide, 0.05% glucose, 0.0255% NaHCO₃, 14 mM HEPES and 4.3 mM sodium pyruvate in Ringer's fluid with pH 7.2 to 7.4). Resulting filtrate was collected into 150 mL Corning tubes filled with 5 mL FBS, which had previously been cooled to 4° C. The filtrate with islets was then centrifuged: 750×g, 2 min, 4° C. Supernatant was removed, and a pellet containing pancreatic islets and digested exocrine tissue was suspended in 100 mL Store Protect solution (UW solution). In order to count isolated islet equivalents, 100 μl of the islet suspension sample was collected and 2 to 3 drops of 0.1% dithizone were added.

Islets were counted in a standardised manner, converting the results obtained to IEQ number.

### 3) Optional Purification of Isolated Islets

In order to purify pancreatic islets, isolated islets were striped of exocrine tissue using Histopaque as reagent.
The islet pellet was suspended in a 10 mL Histopaque and the pellet was gently broken up, followed by another 5 mL Histopaque being added to the suspended pellet; all reagents were at room temperature
15 mL HBSS (without FBS) was added dropwise onto the layer of islets in Histopaque
This was gradient purified, centrifuged for 24 minutes at 2100 rpm
After centrifugation, the entire Histopaque layer was collected from the pellet (approx. 15 mL)
The collected layer was transferred to a 50 mL Falcon tube and filled up with 50 mL HBSS+10% FBS
This was centrifuged at 1000 rpm for 2 min.
Supernatant was removed to leave a final volume of 30 mL and filled with fresh HBSS+10% FBS up to 50 mL
This was centrifuged at 1000 rpm for 2 min.
Supernatant was removed to leave a final volume of 20 mL and filled up with fresh HBSS+10% FBS up to 50 mL
This was centrifuged at 1000 rpm for 2 min.
Supernatant was removed to leave a final volume of 10 mL and filled with fresh HBSS+10% FBS up to 50 mL
This was centrifuged at 1000 rpm for 2 min.
Supernatant was removed to leave a final volume of 5 mL
In this volume, the islets were suspended
The islets obtained were counted on a weigh
The islets alone were collected into a 10 mL Falcon tube
This was centrifuged for 2 min at 1000 rpm

Alternatively, the islets can be treated using the COBE device. This also involves gradient purification (using Islet gradient 1.108 and 1.069 or another gradient solutions).

### Preparing the Sample for Testing

In order to test thermal stability of the samples in various storage conditions, suitable culture media were prepared and 3000 iEQ pancreatic islets per sample were suspended in 3 mL of medium. Samples were incubated at a test temperature for 120 hours, performing sampling every 24 hours, and the GSIS test was performed in order to assess islet functionality.

In all embodiments, 3 mL of commercially available CMRL 1066 base medium (CMRL 1066 with L-glutamine, without sodium bicarbonate, Sigma-Aldrich, Inc.
https://www.sigmaaldrich.com/deepweb/assets/sigmaaldrich/product/documents/254/3 08/c0422dat.pdf) was used.

In each case, for both test and control samples, CMRL 1066 medium was supplemented with additional ingredients (per 500 mL of medium) before use:
L-glutamine—5 mL
100× Penicillin/streptomycin—5 mL
Amphotericin B—5 mL
Glucose 280 mM—10 mL
FBS—50 mL

### 4) Assessing Islet Functionality

Islet functionality was assessed using a glucose-stimulated insulin secretion (GSIS). Solutions of 2.5 and 16.7 mM glucose were prepared by diluting glucose in sterile Krebs buffer (25 mM HEPES, 115 mM NaCl, 24 mM NaHCO₃, 5 mM KCl, 1 mM MgCbx6H₂O, 0.1% BSA, 2.5 mM CaCl₂×2H₂O) and filtered through a 0.22 μm filter. The solutions were previously heated to 37° C. and zero samples were collected from both solutions. 3000 IEQs were placed in each insert and immersed in 2.5 mM glucose solution. After 30 minutes, the inserts were dried on tissue paper and placed in 16.7 mM glucose solution for 60 minutes, dried and placed in a 2.5 mM glucose solution for another 60 minutes.

Samples of 100 μL each were collected every 15 min. Throughout the testing, plates were stored in a cell culture incubator at 37° C., and all samples collected during the GSIS test were frozen at −80° C. before performing the ELISA test. The ELISA test was performed using the Insulin ELISA kit.

### Example 1—Storing Pancreatic Islets at 4° C. and 37° C. in a Medium Containing an ECM Preparation in a Form of a dECM Solution

Media with concentrations of 1% and 2.5% of ECM preparation, respectively, were obtained by blending the following ingredients:

| Final medium | Final ECM | CMRL 1066 | 5% dECM | 10% dECM |
|---|---|---|---|---|
| volume | concentration | medium | solution* | solution* |
| 3 mL | 1% | 2.4 mL | 0.6 mL | |
| | 2.50% | 2.25 mL | | 0.75 mL |

*dECM solution at a suitable concentration was prepared as described in patent application EP19218191

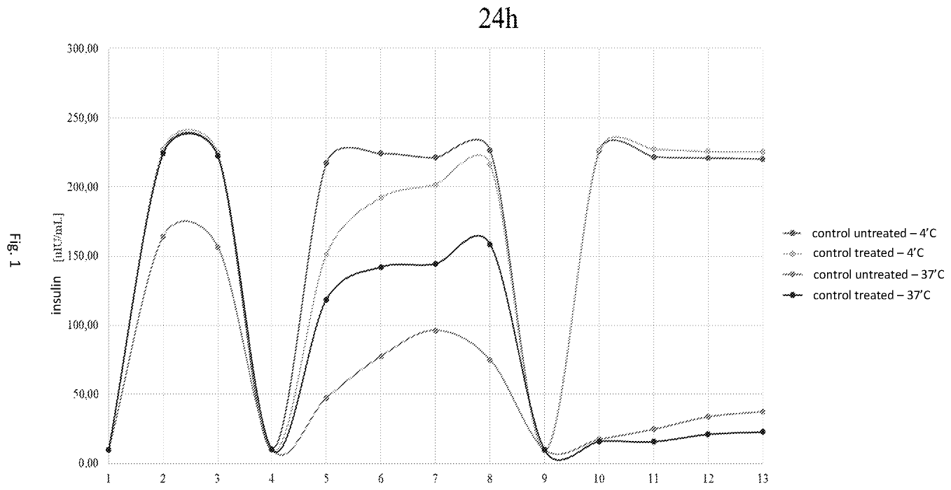

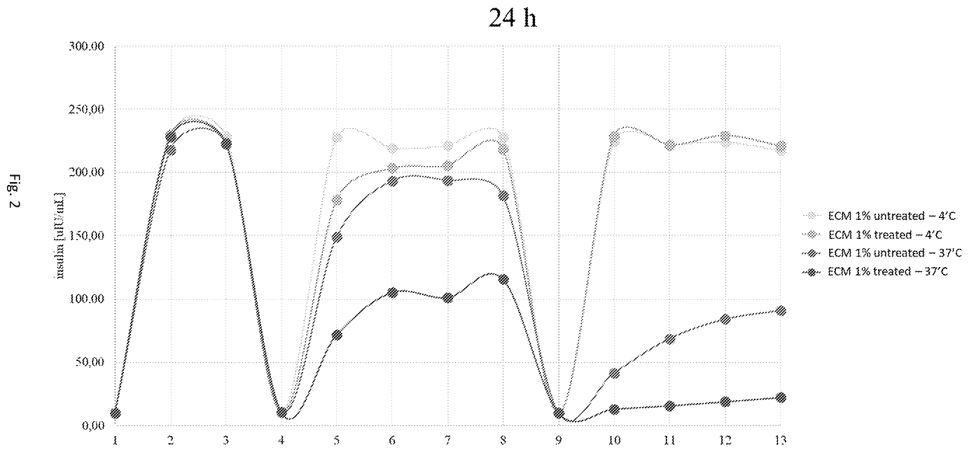

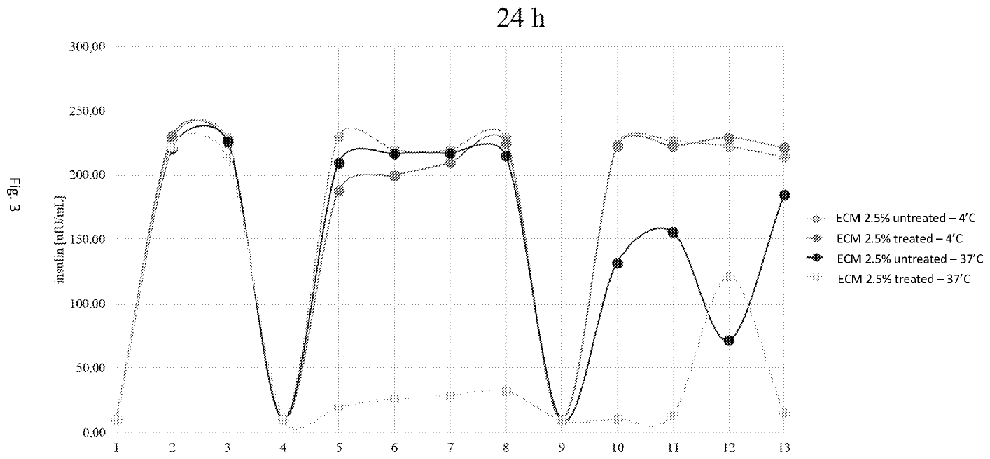

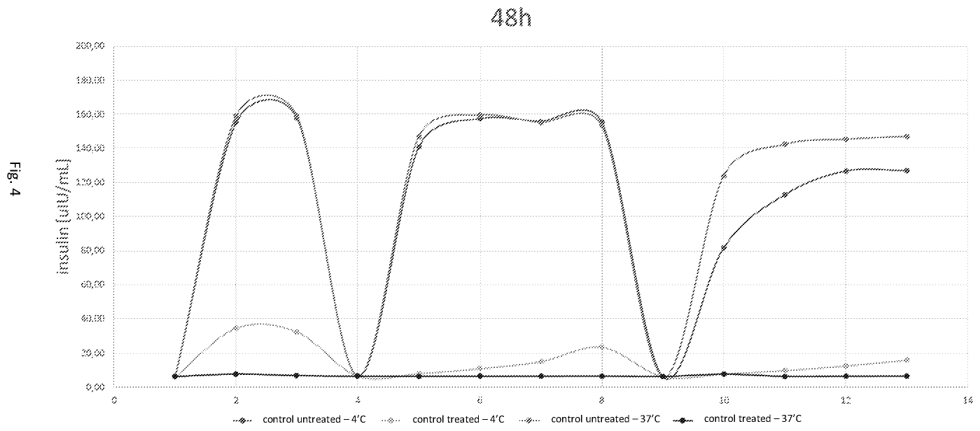

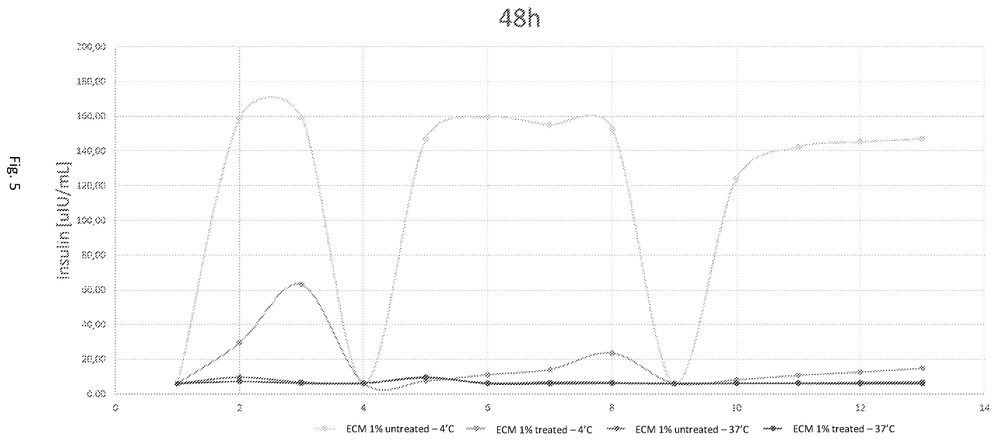

In order to carry out the experiment, 3 mL of each medium was mixed with treated and untreated pancreatic islets and incubated for 120 hours. Results of the experiment are shown in FIG. 1-5. A control sample was CMRL1066 medium without additives.

The addition of ECM had a significant impact on pancreatic islet functionality. A similar effect was observed with reduced storage temperature and using untreated islets directly after isolation.

Resulting graphs clearly show that the functionality of pancreatic islets was more than twice lower when stored for 24 h at 37°° C. than at 4° C. It can also be observed that the islet functionality was superior when they were untreated (after 24 h storage at 4° C., the activity of untreated islets was nearly 100 units higher than that of treated islets).

Also note that the addition of ECM had a significantly impact on the pancreatic islet function. The results obtained for 2.5% ECM are 15% higher than the results for the control sample, but lower than those obtained with 1% ECM preparation. Thus, optimal conditions for storing pancreatic islets are as follows: an ECM preparation content of 1% in CMRL1066 commercial medium, a temperature of 4° C., and untreated islets.

### Example 2—Storing Pancreatic Islets at 4° C. in a Medium Containing an ECM Preparation in a Form of an ECM Protein Mixture

Media containing the mixture of ECM protein components were obtained by blending following components:

| Final | Medium | | Hyaluronic | | |
|---|---|---|---|---|---|
| medium | CMRL | Laminin | acid** | Collagen I | Collagen IV |
| volume | 1066 | (1.0 mg/mL) | (5.0 mg/mL) | (1.0 mg/mL) | (1.0 mg/mL) |
| 3 mL | 2.25 mL | 0.253 mL | 0.004 mL | 0.122 mL | 0.367 mL |

**The testing was conducted in two variants - with high- and low molecular weight hyaluronic acid. The results for both variants were identical.

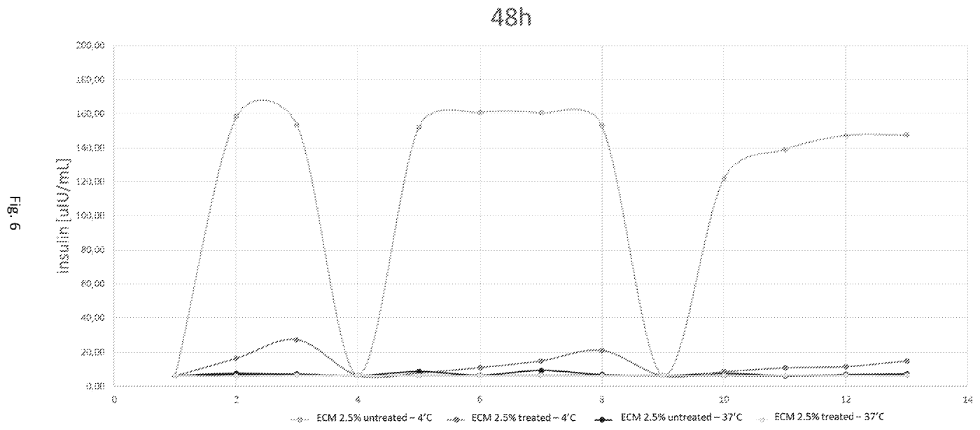

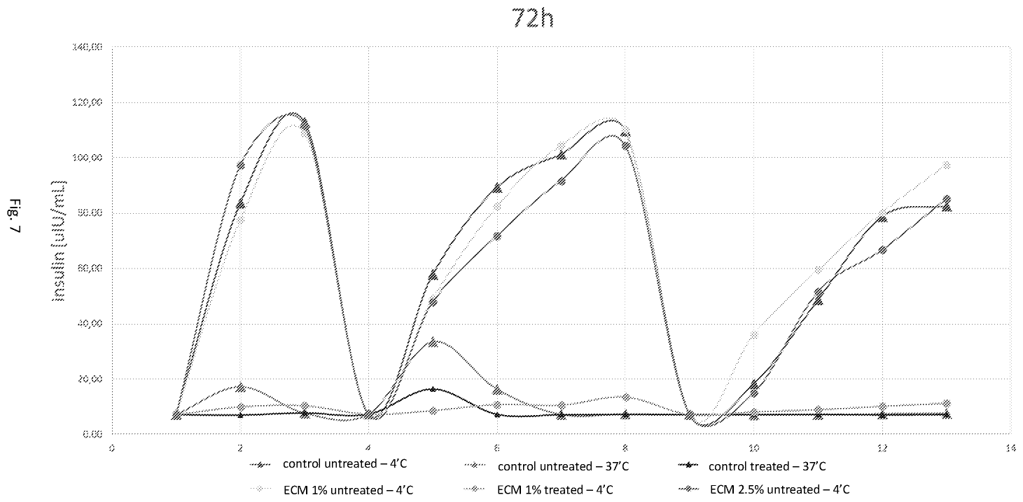

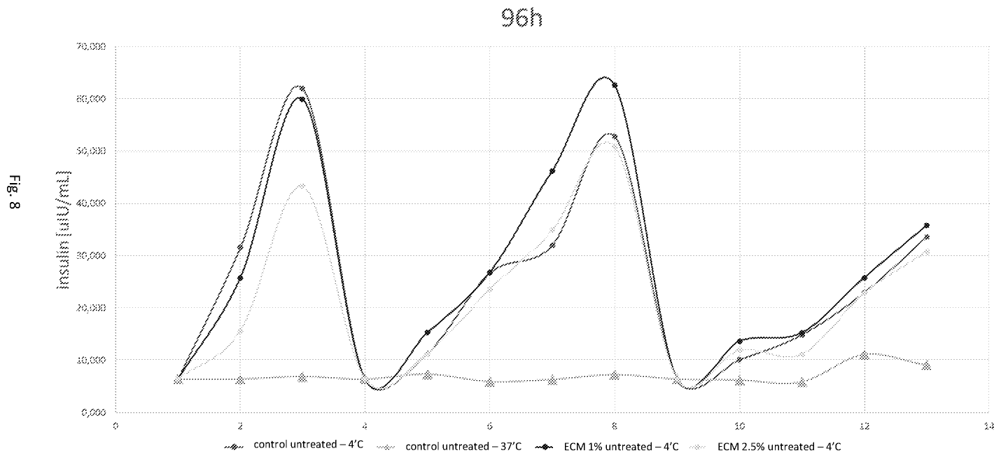

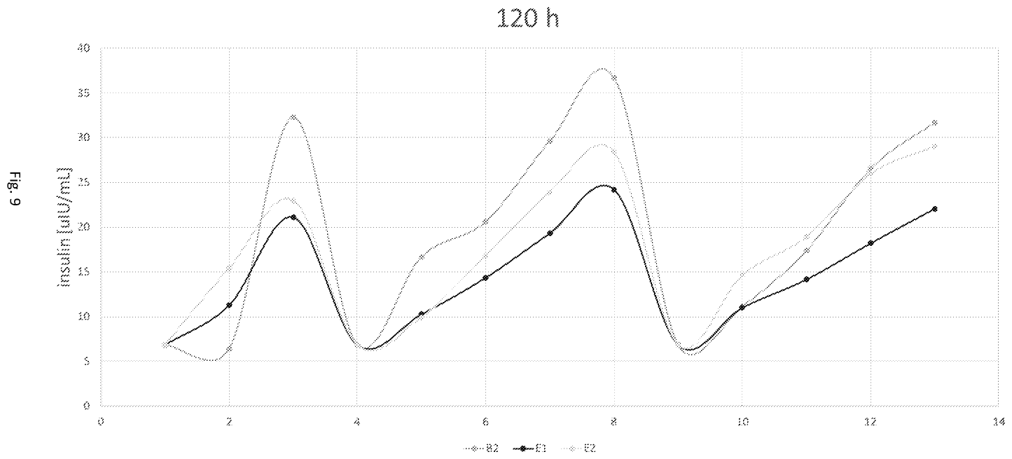

In order to carry out the experiment, 3 mL of the medium was mixed with untreated pancreatic islets and incubated at 4° C. for 72 hours, respectively. The results of the experiment are shown in FIG. 6. The control sample was CMRL1066 medium without additives.

After 72 hours of incubation, an increase in the function of pancreatic islets stored in the medium according to the invention was observed compared to the control sample.

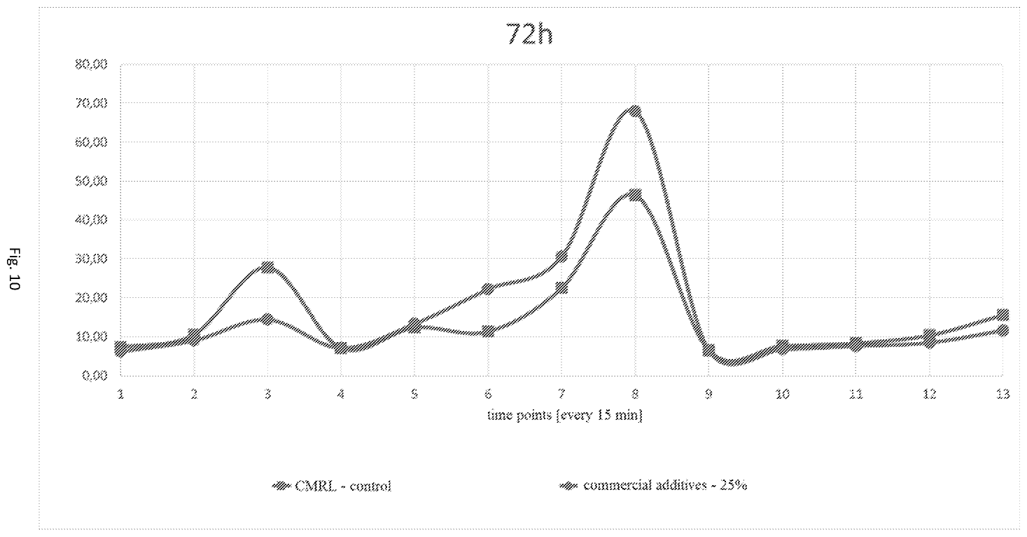

There were no differences between high-and low molecular weight hyaluronic acid (FIG. 10 shows an average result).

1. An isolated pancreatic islet storage medium based on a commercial medium CMRL 1066 or RPMI, characterised in that it comprises an additive of extracellular matrix (ECM) proteins comprising the following proteins in appropriate amounts by weight per 1L of medium:
laminin in an amount of 67 to 100 mg/L of medium,
hyaluronic acid in an amount of 5 to 8 mg/L of medium.
collagen I in an amount of 32 to 48 mg/L of medium,
collagen IV in an amount of 100 to 145 mg/L of medium.

2. The medium according to claim 1, wherein the base is the commercial medium CMRL 1066.

3. The medium according to claim 1, wherein the additive of ECM proteins comprises laminin in an amount of 84.3 mg/L of medium, hyaluronic acid in an amount of 6.7 mg/L of medium, collagen I in an amount of 40.7 mg/L of medium, and collagen IV in an amount of 122.3 mg/L of medium.

4. The medium according to claim 3, wherein the additive of ECM proteins comprises the following proteins in appropriate % by volume relative to the volume of the medium:

| | |
|---|---|
| laminin | 6.74-10.12%, preferably 8.43% by volume of the medium, |
| hyaluronic acid | 0.10-0.16%, preferably 0.13% by volume of the medium, |
| collagen I | 3.26-4.88%, preferably 4.07% by volume of the medium; and |
| collagen IV | 9.78-14.67%, preferably 12.23% by volume of the medium, |

wherein laminin at a concentration of 1 mg/mL, hyaluronic acid at a concentration of 5 mg/mL, collagen I at a concentration of 1 mg/mL, and collagen IV at a concentration of 1 mg/ml are added to the medium.

5. The medium according to claim 3, wherein the hyaluronic acid may be a high or low molecular weight hyaluronic acid.

6. The medium according to claim 1 wherein the medium further comprises a cryoprotectant and/or an antibiotic and/or a fungicide and/or glucose and/or a serum and/or an amino acid.

7-12. (canceled)

13. The medium according to claim 6 comprising L-glutamine, preferably in an amount of 5 mL for each 500 ml of base medium, 100× Penicillin-streptomycin, preferably in an amount of 5 mL for each 500 ml of base medium, amphotericin B, preferably in an amount of 5 mL for each 500 mL of base medium, glucose, preferably in an amount equivalent to 10 mL of 280 mM glucose solution for each 500 mL of base medium, and FBS, preferably in an amount of 50 mL for each 500 ml of base medium.

14. Use of the medium defined claim 1 for storing isolated pancreatic islets at a temperature between 4 and 37° C., preferably at 4° C.

15. The use according to claim 14, characterised in that before the islets are placed in the medium, the islets are treated or they remain untreated.

16. An isolated pancreatic islet storage medium based on a commercial medium CMRL 1066 or RPMI, characterised in that it comprises an additive of extracellular matrix (ECM) proteins in form of a detergent-free decellurized ECM (dECM) solution in an amount of 1% to 2.5% by volume of the medium.

17. The medium according to claim 16, wherein the detergent-free dECM solution is of animal origin.

18. The medium according to claim 16, wherein the detergent-free dECM solution content is 1% by volume of the medium.

19. The medium according to claim 16, wherein the medium further comprises a cryoprotectant and/or an antibiotic and/or a fungicide and/or glucose and/or a serum and/or an amino acid.

20. The medium according to claim 19 comprising L-glutamine, preferably in an amount of 5 mL for each 500 ml of base medium, 100× Penicillin-streptomycin, preferably in an amount of 5 mL for each 500 mL of base medium, amphotericin B, preferably in an amount of 5 mL for each 500 mL of base medium, glucose, preferably in an amount equivalent to 10 mL of 280 mM glucose solution for each 500 ml of base medium, and FBS, preferably in an amount of 50 mL for each 500 mL of base medium.

21. Use of the medium defined in claim 16 for storing isolated pancreatic islets at a temperature between 4 and 37° C., preferably at 4° C.

22. The use according to claim 21, characterised in that before the islets are placed in the medium, the islets are treated or they remain untreated.
